(19) United States
(12) Patent Application Publication
(10) Pub. No.: US 2019/0161737 A1
(43) Pub. Date: May 30, 2019

(54) PROCESS FOR CONTINUOUS CELL CULTURE OF CANCER CELLS AND CANCER STEM CELLS

(71) Applicant: Georgetown University, Washington, DC (US)

(72) Inventors: Maria Laura Aventaggiati, Kensington, MD (US); Giuseppe Giaccone, Bethesda, MD (US)

(73) Assignee: Georgetown University, Washington, DC (US)

(21) Appl. No.: 16/320,850

(22) PCT Filed: Jul. 26, 2017
(86) PCT No.: PCT/US2017/043885

§ 371 Date: Jan. 25, 2019

Related U.S. Application Data
(60) Provisional application No. 62/367,305, filed on Jul. 27, 2016.

(51) Int. Cl. C12N 5/095; C12N 5/00; C12Q 1/02; C12Q 1/6886

(52) U.S. Cl. CPC C12N 5/0695; G01N 2800/52; C12Q 1/025; C12Q 1/6886; C12N 2500/32; C12N 2501/113; C12N 2501/115; C12N 2501/11; C12N 2501/105; C12N 2501/33; C12N 2533/52; C12N 2533/54; C12N 2533/90; G01N 2800/7028; C12N 5/0037

(57) ABSTRACT

The present invention is directed towards compositions and methods of culturing cancer cells, with the methods comprising culturing cancer cells in the presence a cell culture medium while inhibiting the activity of Rho kinase (ROCK) in the cells during culturing.

### STATEMENT REGARDING FEDERALLY SPONSORED RESEARCH OR DEVELOPMENT

Part of the work performed during development of this invention utilized U.S. Government funds under National Institutes of Health Grant No. R01CA193698-01. The U.S. Government has certain rights in this invention.

### BACKGROUND OF THE INVENTION

### Field of the Invention

The present invention is directed towards compositions and methods for culturing cancer cells, with the methods comprising culturing the cells a cell culture medium while inhibiting the activity of Rho kinase (ROCK) in the cells during culture. The present invention is also directed towards methods of using these cultured cancer cells.

### BACKGROUND OF THE INVENTION

It has been difficult to consistently expand Cancer Stem Cells (CSCs) isolated from patients for phenotypic characterization, since they not only represent a small cell sub-population, but also undergo differentiation in canonical culturing conditions. It would invaluable to obtain and propagate a collection of CSCs from patient-derived tumor samples for potential real-time drug screening and molecular, phenotypic and functional characterization. The present application provides a protocol that captures and propagates cultures of CSCs in a reasonable time frame, in some cases within one week of placing the specimens in culture.

### SUMMARY OF THE INVENTION

The present invention is directed towards compositions and methods for culturing cancer cells, with the methods comprising culturing the cells a cell culture medium while inhibiting the activity of Rho kinase (ROCK) in the cells during culture. The present invention is also directed towards methods of using these cultured cancer cells.

The present invention is also directed towards methods of producing conditionally immortalized cancer cells, with the methods comprising culturing the cells in a cell culture medium while inhibiting the activity of Rho kinase (ROCK) in the cells during culture. Culturing the cancer cells in such conditions will produce conditionally immortalized cancer cells.

### BRIEF DESCRIPTION OF THE DRAWINGS

FIG. 1 depicts the structures of select ROCK inhibitors.

FIG. 2A-C depicts various micrographs of captured, cultured cancer cells using the methods and cell culture compositions and/or systems of the present invention. 2A shows patient-derived tumor cells after 48 hours of placing them in GMSC matrix. In about 7 days, a sizeable pellet of cells was obtained. The middle insert in 2A is a magnification of the rectangle shown in left panel of 2A. The right panel is a differential interference contrasting (DIC) microscope image of the same tumor culture. 2B shows the same amount of tumor cells as in A, except placed in standard cell culture plates without a matrix and grown in Dulbecco Modified Eagle media (DMEM) in 10% FBS, as per standard laboratory procedure. 2C shows approximately the same amount of material as in A that was placed in a cell culture environment with ROCK inhibitor and feeder cells, but not in the conditions of the present invention.

FIG. 3A-F depicts the expansion of primary cells isolated from pleural effusion of a patient with metastatic lung NSCLC. 3A-3B show bright field and DIC images of primary cells placed in matrix-coated plates and grown in GMSC media. 3C shows cells from the same specimen were placed in culture in standard tissue culture plates in DMEM media in the presence of 10% serum. 3D-3F show different fields and magnifications of a pleural effusion (total volume 40 ml), at 36 hours after placing it in culture in GMSC matrix.

FIG. 4A-F depicts three different established tumor cell line (MP25, MP31 and MP39) using the methods and compositions of the present invention. This culture was established in GMSC and subsequently cells were adapted to grow in standard culturing conditions. 4A shows MP31 cells in DMEM and 10% FBS in standard cell culture plates. 4B-4C show MP31 cells propagated in GMSC at two different magnifications. 4D-4E show MP39 cells, established from a primary NSCLC tumor using GMSC conditions and shown at two different magnifications. 4F shows MP25 cells, established from a third tumor specimen.

FIG. 5A-B depicts flow cytometry analysis for stem cell markers, CD166 and CD133 in MP31 grown in canonical conditions, in the presence of serum (DMEM) or in a cell culture system of the present invention. There is a significant enrichment of CSCs in the novel culture conditions prescribed herein, but not in typical DMEM.

FIG. 6A-G depicts SLC25A1-dependent mitochondrial-respiration driving therapy resistance in patient-derived tumors. FIG. 6A-B: MP1 and MP2 cells were treated with cisplatin (0.5-1 mM) or AZD9291 (1 mM), as indicated in each panel, passaged in the presence of the drugs, and then subjected to OCR analysis with the Seahorse analyzer. FIG. 6C: FACS analysis of CD166 and CD133 markers in the cisplatin or AZD9291 resistant MP1 or MP2 cells. FIG. 6D-G: MP1 (e), MP2 (f) MP3 (g) and MP4 (h) cells were untreated or treated with the indicated drugs. The concentration of drugs employed is indicated at the bottom of each panel. Viability was assessed with crystal violet after 5 days of treatment. Relative (R) index calculations were used to assess the type of drug interactions and are indicated in each panel. The R index was calculated as the expected cell survival (Sexp; the product of relative survival in cisplatin and relative survival in CTPI-2) divided by the observed relative survival in the presence of both drugs (Sobs). Sexp/Sobs=1.0 denotes an additive interaction, while>1.0 denotes a synergistic interaction, R index values approaching 2.0 are indicative of strong synergy.

### DETAILED DESCRIPTION OF THE INVENTION

The present invention is directed towards compositions and methods for culturing cancer cells, with the methods comprising culturing the cells a cell culture medium while inhibiting the activity of Rho kinase (ROCK) in the cells during culture. The present invention is also directed towards methods of using these cultured cancer cells.

As used herein, the term “cancer cell” refers to a cell or cells that are obtained from abnormal tissues or conditions such as, but not limited to, hypertrophy, neoplasia, hyperplasia, benign and malignant tumors. As used herein, the term “tumor” is a general term that includes hypertrophies, neoplasias, hyperplasias, benign cancers and malignant cancers. Accordingly, certain embodiments of the present invention include compositions and methods useful for isolating and/or growing cells isolated from a hypertrophy, a neoplasia, a hyperplasia, a benign or a malignant cancer in a subject. Other types of cancer cells include abnormal cells obtained from blood-born cancers (or non-solid tumors), such as lymphomas, leukemias and the like.

The cancer cells can be from any animal, including but not limited to any mammal, such as mouse, rat, canine, feline, bovine, equine, porcine, non-human and human primates. Mammalian cells particularly suitable for cultivation in the present media include cancer cells of human origin. In addition, transformed cells or established cell lines cancer cell lines can also be used. In one embodiment, the cells are primary or secondary cancer cells. In another embodiment, the cells are not primary cells, such as cells from an established cell line, transformed cells, thawed cells from a previously frozen collection and the like. Animal cells for culturing by the present invention may be obtained commercially, for example from ATCC (Rockville, Md.), Cell Systems, Inc. (Kirkland, Wash.), Clonetics Corporation (San Diego, Calif.), BioWhittaker (Walkersville, Md.) or Cascade Biologicals (Portland, Oreg.).

As used herein, primary cancer cells are cells that have been taken directly from living tissue, such as a biopsy, and have not been passaged or only passaged one time. Thus, primary cells have been freshly isolated, often through tissue digestion and plated. Provided the cells have been passaged one time or less, primary cells may or may not be frozen and then thawed at a later time. In addition, the tissue from which the primary cancer cells are isolated may or may not have been frozen or preserved in some other manner immediately prior to processing.

By “cell culture” or “culture” is meant the maintenance of the cells in an artificial, in vitro environment. The term “cell culture” also encompasses cultivating individual cells and tissues.

The cell seeding densities for each experimental condition can be manipulated for the specific culture conditions needed. For routine culture in plastic culture vessels, an initial seeding density of from about 1×10⁴ to about 1×10⁷ cells per cm² is fairly typical, e.g., 1×10⁶ cells are often cultured in a 75 cm² culture flask. Using the methods of the present invention, however, even a single cell can be plated initially. Thus, the methods of the present invention can be performed using 1, 2, 3, 4, 5, 6, 7, 8, 9, 10, 20, 30, 40, 50, 60, 70, 80, 90, 100 or more cells for an initial cell seeding. Of course, higher cell seeding numbers can be used, such as but not limited to 1×10³, 1×10⁴, 1×10⁵ and so on. Cell density can be altered as needed at any passage.

Mammalian cells are typically cultivated in a cell incubator at about 37° C. at normal atmospheric pressure. The incubator atmosphere is normally humidified and often contain about from about 3-10% carbon dioxide in air. Temperature, pressure and CO₂ concentration can be altered as necessary, provided the cells are still viable. Culture medium pH can be in the range of about 7.1 to about 7.6, in particular from about 7.1 to about 7.4, and even more particular from about 7.1 to about 7.3.

According to the methods of the present invention, cancer cells are cultured in novel cell culture compositions. The cell culture compositions of the present invention comprise fibroblast growth factor (FGF), epithelial growth factor (EGF), insulin growth factor-1 (IGF-1), insulin, progesterone, transferrin, putrescine, pyruvate, albumin, selenite, thiamine, glutathione, ascorbic acid, and at least one Rho kinase (ROCK) inhibitor.

In select embodiments, the cell culture compositions of the present invention comprise glucose. In a more specific embodiment, the cell culture compositions of the present invention comprise high levels of glucose. In more specific embodiments, the cell culture compositions of the present invention comprise glucose at concentrations of between about 2 mM and about 50 mM. In even more specific embodiments, the cell culture compositions of the present invention comprise glucose at concentrations of between about 5 mM and about 45 mM, between about 10 mM and about 40 mM, between about 15 mM and about 35 mM, between about 20 mM and about 30 mM or between about 22 mM and about 28 mM. In one specific embodiment, the cell culture compositions of the present invention comprise glucose at concentrations of about 25 mM.

In additional select embodiments, the cell culture compositions of the present invention comprise at least one amino acid. In more specific embodiments, the cell culture compositions of the present invention comprise at least one amino acid selected from the group consisting of glycine, histidine, isoleucine, methionine, phenylalanine, proline, hydroxyproline, serine, threonine, tryptophan, tyrosine and valine.

In additional select embodiments, the cell culture compositions of the present invention do not contain or comprise animal serum.

The cell culture compositions of the present invention are generally composed of a base cell culture medium. For example, select embodiments of the present invention include but are not limited to cell culture compositions comprising one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM).

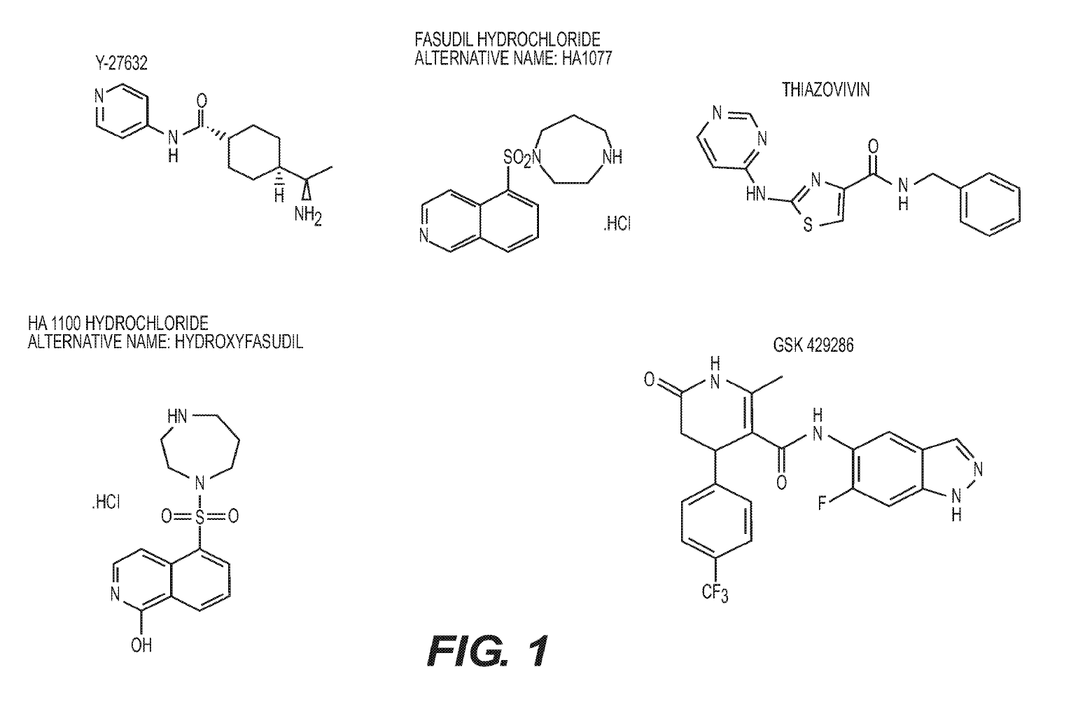

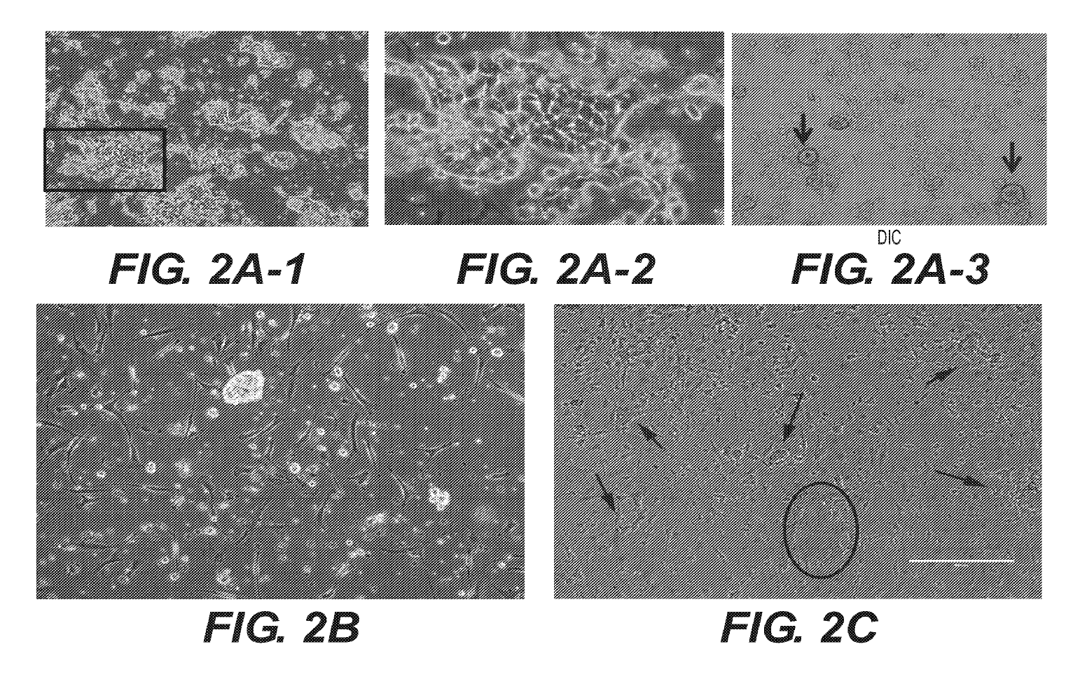

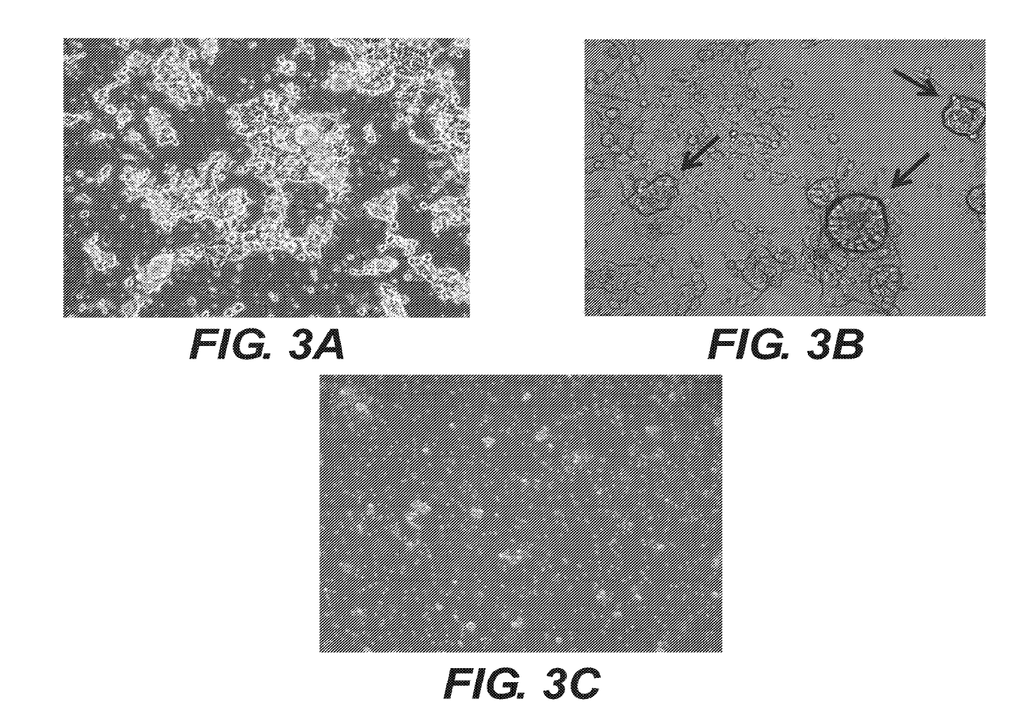

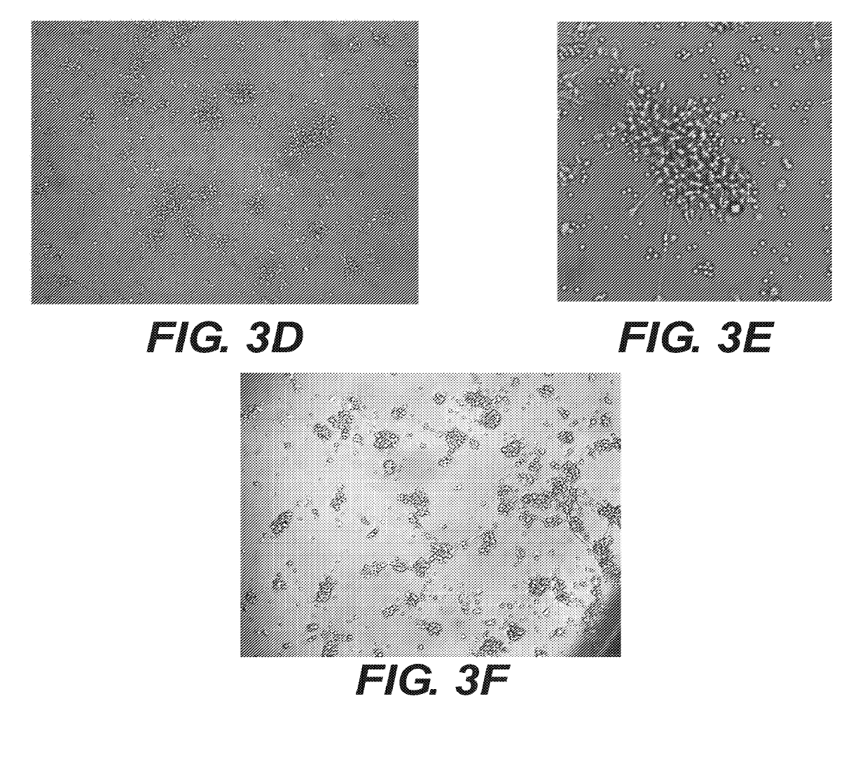

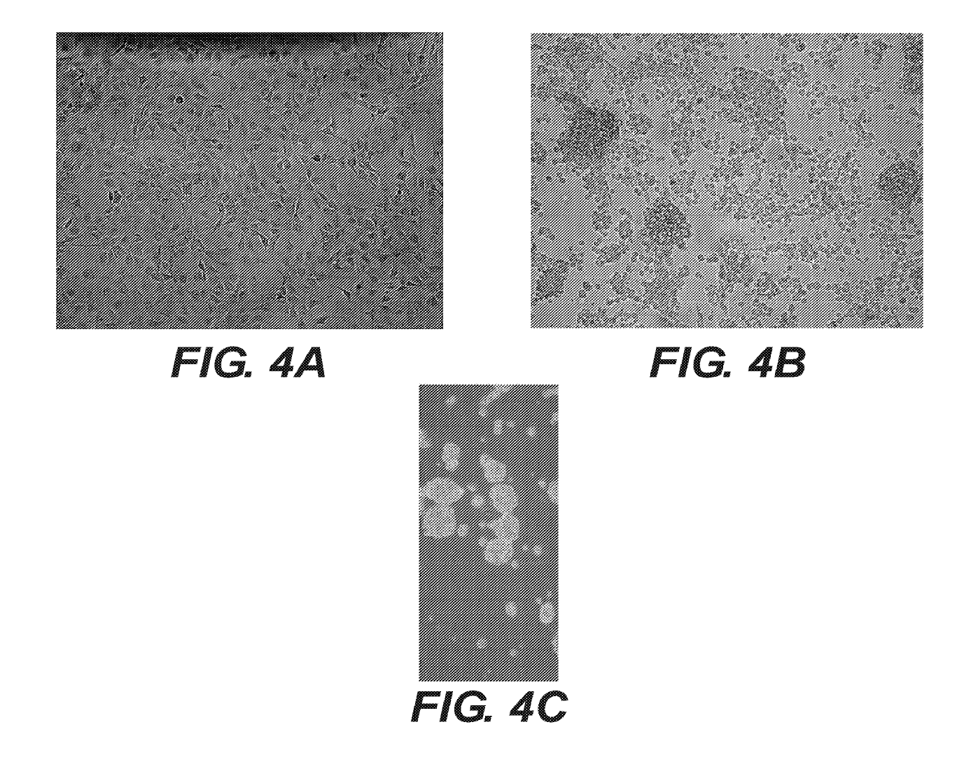

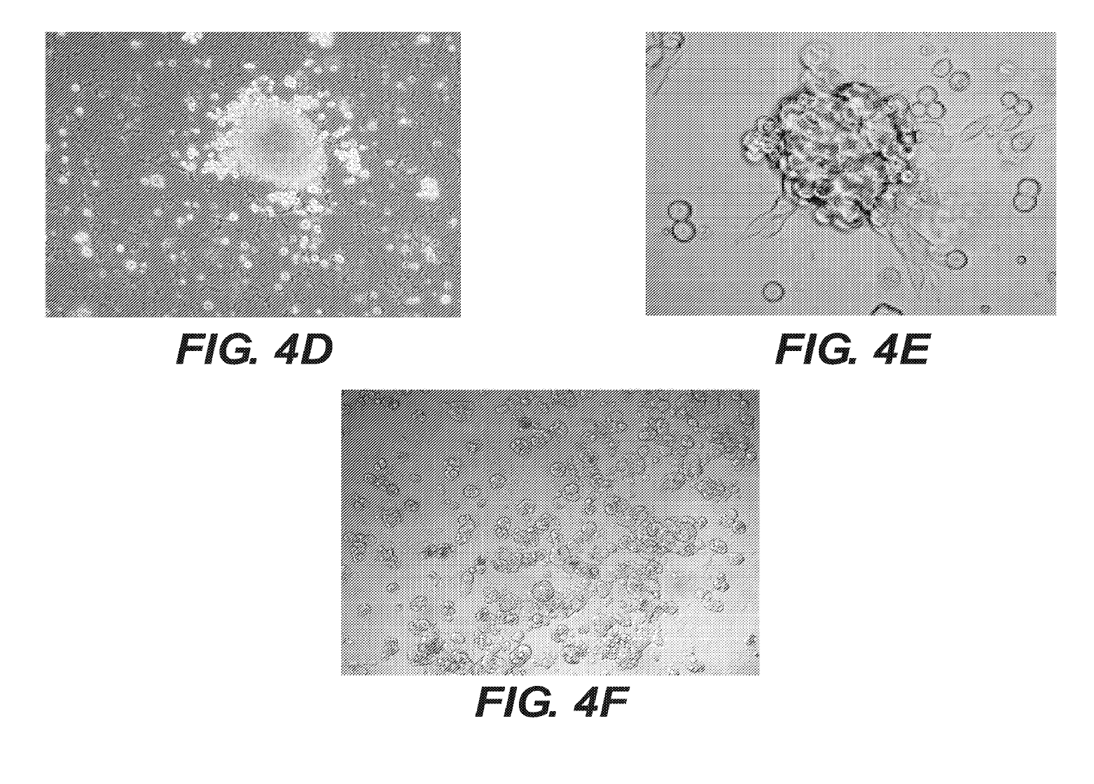

In additional select embodiments, the cell culture compositions of the present invention comprise at least one inhibitor of Rho kinase inhibitor 1 (ROCK 1), Rho kinase inhibitor 2 (ROCK 2) or both. Examples of ROCK inhibitors include but are not limited to Y-27632, HA1100, HA1077, Thiazovivin and GSK429286, the structures of which are depicted in FIG. 1. These compounds are well known and commercially available. Additional small molecule Rho kinase inhibitors include but are not limited to those described in PCT Publication Nos. WO 03/059913, WO 03/064397, WO 05/003101, WO 04/112719, WO 03/062225 and WO 03/062227, and described in U.S. Patent Nos. 7,217,722 and 7,199,147, and U.S. Patent Application Publication Nos. 2003/0220357, 2006/0241127, 2005/0182040 and 2005/0197328, the contents of all of which are incorporated by reference. In select embodiments, the cell culture compositions of the present invention comprise at least one ROCK inhibitor selected from the group consisting of Y-27632, HA1100, HA1077, Thiazovivin and GSK429286.

Another way of inhibiting ROCK kinase would be through the use of RNA interference (RNAi). RNAi techniques are well known and rely of double-stranded RNA (dsRNA), where one stand of the dsRNA corresponds to the coding strand of the mRNA that codes for ROCK1 and/or ROCK2, and the other strand is complementary to the first strand. The requirements of optimal RNAi species for a given nucleotide sequence are well-known or can be readily ascertained given the state of the art. For example, it is known that optimal dsRNA is about 20-25 nt in length, with a 2 base overhand on the 3′ end of each strand of the dsRNA, often referred to as short interfering RNAs (siRNA). Of course, other well-known configurations such as short hairpin RNA (shRNA) may also work. shRNAs are one continuous RNA strand where a portion is self-complementary such that the molecule is double-stranded in at least one portion. It is believed that the cell processes shRNA into siRNA. The term RNAi molecule, as used herein, is any double stranded double-stranded RNA (dsRNA), where one stand of the dsRNA corresponds to the coding strand of the mRNA that codes for the target gene to be silenced, and the other strand is complementary to the first strand.

Other vitamins that may be added to the cell culture compositions of the present invention include but are not limited to biotin, choline chloride, D-Ca⁺²-pantothenate, folic acid, i-inositol, niacinamide, pyridoxine, riboflavin, thiamine and vitamin B12.

Inorganic salt ingredients which may be added to the cell culture compositions of the present invention include but are not limited to calcium salt, e.g., CaCl₂, CuSO₄, FeSO₄, KCl, a magnesium salt, e.g., MgCl₂, a manganese salt, e.g., MnCl₂, sodium acetate, NaCl, NaHCO₃, Na₂HPO₄, Na₂SO₄ and ions of the trace elements selenium, silicon, molybdenum, vanadium, nickel, tin and zinc. These trace elements may be provided in a variety of forms, for example in the form of salts such as Na₂SeO₃, Na₂SiO₃, (NH₄)6Mo₇O₂₄, NH₄ VO₃, NiSO₄, SnCl and ZnSO.

The present invention also relates to cell culture systems comprising the cell culture compositions of the present invention and a culture vessel. The culture vessel can be any standard culture vessel, such as a flask, dish, 96-well plate, etc. Any vessel comprising a bottom surface capable of being coated and side walls that contain the culture medium compositions of the present invention. In specific embodiments, the culture vessels in the systems of the present invention comprise extracellular matrix (ECM) components as a coating on one or more surfaces of the vessel, e.g., the bottom, interior surface. In other words, the present invention also relates to three-dimensional cell culture systems. As used herein, a three-dimensional cell culture vessel, or 3D cell culture vessel, is a vessel containing at least one ECM component or at least one component that mimics a natural ECM structure or component such that the cultured cells can interact with their environment in all directions, rather than the typical cell-to-vessel interaction.

In more specific embodiments, the ECM components in the culture vessels are human-derived components. Examples of human-derived ECM components include but are not limited to collagens (collagen I, II, Ill, IV, V, etc.), laminin, fibronectin, tenascin and elastin.

In additional embodiments, the ECM components in the culture vessels comprise ECM components that are not human-derived components, e.g., murine, bovine, canine, non-human primate ECM components. Examples of non-human derived ECM components include but are not limited to entactin, heparan sulfate proteoglycan or a combination thereof. Other non-human ECM components in the culture vessel may include but are not limited to collagens, laminin, elastin and fibronectin.

In one embodiment, the culture vessels can be comprised of only human-derived ECM components. In another embodiment, the culture vessels can be comprised of only non-human-derived ECM components. In still other embodiments, the culture vessels can comprise a mixture of human and non-human derived ECM components. In still another embodiment, any of the ECM components used to produce a 3D culture vessel may or may not be fabricated into hydrogels. Techniques for fabricating hydrogels using natural ECM components are well-known in the art.

The present invention also provides for methods of culturing cancer cells using the culture systems of the present invention. In general, the methods comprise placing cells isolated from biopsies into the cell culture systems of the present invention.

When isolating primary cells from tissue samples, tissue should ideally be handled using standard sterile techniques and a laminar flow safety cabinet. In one embodiment, a single needle biopsy is sufficient to isolate enough primary cells to begin the cell culture methods of the present invention. In the case of a tissue biopsy, tissue can be cut into small pieces using sterile instruments. The small pieces can then be washed several times with sterile saline solution or other buffer, such as PBS, that may or may not be supplemented with antibiotics or other ingredients. After washing, the pieces are often, but need not be, treated with an enzymatic solution such as, but not limited to collagenase, dispase or trypsin, to promote dissociation of cells from the tissue matrix.

Dispase is often used to dissociate various tissue such as but not limited to epithelium. In addition, intact tissue may also be treated with trypsin or collagenase. These digestion steps often results in a slurry containing dissociated cells and tissue matrix. The slurry can then be centrifuged with sufficient force to separate the cells from the remainder of the slurry. The cell pellet can then be removed and washed with buffer and/or saline and/or cell culture medium. The centrifuging and washing can be repeated any number of times. After the final washing, the cells can then be washed with any suitable cell culture medium. Of course, the digestion and washing steps need not be performed if the cells are sufficiently separated from the underlying tissue upon isolation, such as the case in a needle biopsy or if isolated from the circulation. Cells may or may not be counted using an electronic cell counter, such as a Coulter Counter, or they can be counted manually using a hemocytometer. Of course, the cells need not be counted at all.

For the purposes of the present invention cells are no longer considered to be primary cells after the cells have been passaged more than once. In addition, cells passaged once or more and immediately frozen after passaging are also considered not to be primary cells when thawed. In select embodiments of the present invention, the cancer cells are initially primary cells and, through the use of the methods of the present invention, become non-primary cells after passaging.

The present invention also provides for methods of isolating cancer cells from biopsies or tissue specimens, comprising placing the cells in a cell culture system of the present invention. In general, the biopsied material can be isolated and digested, for example applying collagenase, dispase and/or trypsin to the tissue to promote dissociation of cells from the tissue matrix, using standard cell culture procedures. Once the material has been subjected to a procedure intended to dissociate the cells from the underlying matrix, the resulting material may or may not be processed further, for example centrifugation. The processed material that includes the cells can then be plated onto the inventive culture vessels of the present invention in the presence of the inventive cell culture compositions of the present invention. The cell culture vessels of the present invention are used to capture the cancer cells of interest, and the cell culture compositions of the present invention are used to propagate these captured cells.

Subjecting the captured cells to the methods and systems of the present invention will establish a population of conditionally immortalized cancer cells. Accordingly, the present invention provides for a population of conditionally immortalized cancer cells. In one embodiment, the methods and systems of the present invention recapitulate the spatial heterogeneity of cancer cells or cancer tissue in their physiological state. In other words, the methods and systems of the present invention provide cell culture compositions in which the cells are not clonal such that the cancer cell population can potentially contain genetically and/or physiologically heterogenous mixtures of cells.

In another embodiment, the population of immortalized cancer cells is a population of cancer stem cells (CSCs). As used herein, the term cancer stem cells (CSCs) is used to mean cells that have the capacity to propagate and self-renew indefinitely by mean of asymmetric cell division, thus giving rise to one “differentiated” cell and one undifferentiated cell. It is well understood that cancer cells generally represent a less differentiated cell than a normal cell, but cancer cells nonetheless retain at least some of the phenotypic and/or genotypic markers of differentiated normal cells such that the cancer cells can generally be classified according to the tissue of origin, e.g., a breast cancer cell, etc. As used herein, a “differentiated cell,” is a cell that is a differentiated normal cell or a cancer cell that retains at least some of the phenotypic and/or genotypic markers of differentiated normal cells such that the “differentiated cancer cell” can generally be classified according to the tissue of origin. CSCs are likely the source of cancer initiation and metastatic dissemination and can be identified by virtue of specific functional and phenotypic markers. For example, surface markers of CSCs include but are not limited to CD166, CD133, CD44, CD24, CD25, and CSCs also possess the ability to retain specific stains/dyes (Hoechst 33342) and often possess high levels/activity of aldehyde dehydrogenase (ALDH) that can be measured experimentally.

Cell culture medium is normally replaced every 1-2 days or more or less frequently as required by the specific cell type. As the cancer cells approach confluence in the culture vessel, they would normally be passaged. As used herein a cell passage is a term that is used as it is in the art and means splitting or dividing the cells and transferring a portion of the cells into a new culture vessel or culture environment. Most likely, the cancer cells used in the methods of the present invention will be adherent to the cell culture surface and will need to be detached. Methods of detaching adherent cells from the surface of culture vessels are well-known and commonly employed and can include the use of enzymes such as trypsin.

A single passage refers to when a technician splits or manually divides the cells one time and transfers a smaller number of cells into a new vessel or environment. When passaging, the cells can be split into any ratio that allows the cells to attach and grow. Thus, at a single passage the cells can be split in a 1:2 ratio, 1:3, 1:4, 1:5 etc. Passaging cells, therefore, is not necessarily equivalent to population doubling. As used herein a population doubling is when the cells divide in culture one time such that the number of cells in culture is approximately doubled. Cells need to be counted to determine if a population of cells has doubled, tripled or multiplied by some other factor. In other words, passaging the cells and splitting them in a 1:3 ratio for further culturing in vitro is not to be taken as the equivalent that the cell population has tripled.

In one embodiment of the present invention, the cancer cells are continuously cultured in vitro. As used herein, “continuous culturing” is the notion that the cells continually divide and reach or approach confluence in the cell culture vessel such that the cells require passaging and fresh medium to maintain their health. Thus, the concept of “continuously culturing” is similar to the concept that the cancer cells would be “immortalized.” Accordingly, the present invention is also directed to conditionally immortalized cancer cells, with the term “conditionally immortalized” referring to the ability of the cells to divide in the prescribed culture conditions indefinitely, i.e., regardless of the number of passages, such that the cancer cells growing in the prescribed conditions would need to be passaged to maintain their health. In one embodiment, when cultured using the present methods and conditions of the present invention, cancer cells can continue to grow and divide for at least 5, 10, 15, 20, 25, 30, 35, 40, 45, 50, 55, 60, 65, 70, 75, 80, 85, 90, 95, 100, 125, 150, 175, 200, 250 or 300 passages or more.

The present invention is also directed towards methods of stimulating growth of cancer cells in vitro with the methods comprising culturing the cancer cells in the cell culture compositions and/or cell culture systems of the present invention. Culturing the cancer cells in such conditions will stimulate the cancer cells to grow or proliferate, whereas otherwise the cancer cells may not grow.

As used herein and throughout the specification, “cell growth” refers to cell division, such that one “mother cell” divides into two “daughter cells.” As used herein, “cell growth” does not refer to an increase in the actual size of the cells. Stimulation of cell growth can be assayed by plotting cell populations over time. A cell population with a steeper growth curve can said to be growing faster than a cell population with a curve not as steep. Growth curves can be compared for various treatments between the same cell types, or growth curves can be compared for different cell types.

Currently acceptable or optimal conditions for culturing cancer cells generally include culturing cells in well-defined, or synthetic, serum-free medium. For example, culturing cancer cells normally involves culturing in cell-specific medium, with added serum or tissue specific growth factors. Thus, “currently acceptable” or “currently optimal” culture conditions are culture conditions where the medium includes serum or a serum replacement. “Currently acceptable” or “currently optimal” culture conditions may also include the use of synthetic or well-defined medium, for example the use of mammary cell-specific cell medium for mammary cancer cells.

The present invention also provides for methods of identifying a candidate cancer treatment for a subject in need of a treatment thereof, the method comprising obtaining cells isolated from a biopsy from the subject, culturing the isolated cells in one of the cell culture systems or compositions of the present invention, to produce a population of cancer cells in vitro. Once established, the population of cancer cells can then be used to develop a response profile of at least a portion of the cancer cells in vitro, and identifying a candidate treatment for the subject based on the determined response profile.

A response profile, as used herein, is a collection of one or more data points that would indicate, e.g., to a clinician, the likelihood that a particular treatment will produce a desired response in the cancer cells if they were in an in vivo setting. A “response” as used in connection with a response profile may or may not be either cell death by any means (necrosis, toxicity, apoptosis etc) or a reduction of the growth rate of the cancer cells. The response profile need not predict a response with 100% accuracy. A response profile can be a single data point or it can be a collection of data.

Any method can be used to identify or determine the response profile of a given population of cancer cells. For example, the response profile may be assessed by sequencing at least part of the DNA or RNA that is isolated from the cancer cells. This may be particularly useful when it is suspected that a virus may be causing the abnormal condition. It is not necessary that all of the DNA/RNA be sequenced to provide at least one data point for the response profile. For example, using well-known techniques involving polymerase chain reaction (PCR), it would currently be a matter of simple procedure to use PCR primers with sequences specific for the DNA/RNA suspected of being present in a PCR reaction to determine if a product is made. If no detectable product is generated after the PCR reaction using specific primers, it may be possible to conclude that the portion of the protein for which the PCR primers are specific may not be present. Likewise, determining the absence of a particular DNA/RNA sequence could also be a data point in a response profile. In this manner, the DNA or RNA is “sequenced” for the purposes of the present invention, although the precise sequence is not determined for the entire DNA/RNA sequence isolated from the cells. Thus, “sequencing” as used herein may or may not result in generating the entire nucleotide sequence of the isolated DNA/RNA. Other methods can also be used to determine the sequence of the isolated DNA/RNA such as, but not limited to Southern blots, Northern blots, RT-PCR, automated sequencing and the like. Methods of sequencing DNA/RNA are well known in the art and need not be repeated herein.

Similarly, the response profile may be assessed by identifying the presence or absence of at least a portion of one mRNA that may be produced in the cancer cells in vitro. Like determining the sequence of the DNA/RNA above, the precise sequence of the mRNA need not be determined for the entire mRNA isolated from the cells. Methods that can also be used to determine the presence or absence of the sequence of the isolated mRNA include but are not limited to Northern blots, RT-PCR, automated sequencing and the like. Methods of identifying the presence or absence of the at least one mRNA are well known in the art and need not be repeated herein.

Similarly, the response profile may be assessed by identifying the presence or absence of at least a portion of one protein that may be produced in the cancer cells in vitro. Like determining the sequence of the DNA/RNA above, the precise amino acid sequence of the present or absent protein need not be determined for the entire protein. Methods that can also be used to determine the presence or absence of the sequence of the isolated protein include but are not limited to Western blots, immunohistochemical methods, ELISA methods, and the like. Methods of identifying the presence or absence of the at least one protein are well known in the art and need not be repeated herein. The presence or absence of a protein, e.g., a receptor, may indicate that the cells are susceptible to a particular treatment that may, for example, result in cell death.

The response profile may be assessed by subjecting the cancer cells in vitro to a chemotherapeutic agent and determining the response of the cells to the chemotherapeutic agent. As used herein, a chemotherapeutic agent is not limited to traditional cancer treatments but is used to indicate a therapeutic treatment of any kind using a chemical entity. In one embodiment, the response to the therapeutic agent can be assessed by determining the therapeutic index of the agent on the cells. Determining the therapeutic index is common in the art and is simply the ratio of the LD₅₀/EC₅₀, with the LD₅₀ representing the median lethal dose and the EC₅₀ representing the half maximal dose of the agent on the cells. Other methods to assess a response to the agent include but are not limited to determining dose response curves, cell survival curves and the like. In one embodiment, the agent that is used to determine the response of the cancer cells to the agent can be the same or a different agent that is later administered to the subject.

The present invention also provides kits for culturing cancer cells and/or generating conditionally immortalized cancer cells. The kits can include culture vessels, culture media in wet or dry form and/or individual media components. The kit may or may not include chemicals, such as trypsin, for passaging cells, etc.

### EXAMPLES

### Example 1

### Harvesting and Culturing of Cancer Cells

Preparation of the Plates Coated with Matrix

The GELTREX™ and the MAXGEL™ were thawed for 1-2 hours on ice. The desired volume of matrix was prepared by working with ice chilled DMEM/F12 (no antibiotics, no supplements at this stage). GELTREX™ is added at a final concentration of 2.5% and MAXGEL™ was added at a final concentration of 1% to the desired volume of DMEM/F12 on ice. The composition is gently mixed to avoid bubbles and a thin layer for coating the plates was used, e.g., for 6 wells plates, about 1.5 ml of matrix mix, for 10 cm plates, about 8 ml is sufficient.

The plates were incubated at 37° C. for about 1.5 hours, or until the polymerization of the matrix was well visible under microscope examination. The plates were then incubated for at least 30 minutes at room temperature, to favor further stabilization of the matrix. The plates can be used immediately or can be stored in a 4° C. refrigerator, wrapped with parafilm or other wrap to prevent evaporation.

Preparation of the Media

The media was prepared as a mixture of DMEM/F12 (Thermo-Fisher #11320-033 basic media, with high glucose, glutamine and pyruvate) supplemented with antibiotics. To the media bottle that was stored at 4° C., the following components were added to prepare a “pre-mix”: Insulin-Transferrin-Sodium selenite (ITS; 1:1000; Sigma #11884), Glutamine (Thermo-Fisher), Sodium Pyruvate (Thermo-Fisher), Rock Inhibitor (Y-27632; 5-10 μg/ml), between about 2-10% KNOCK OUT SERUM REPLACEMENT (KNOW;Thermo-Fisher #10828-028), 1% ALMUMAX-(LIPID-ENRICHED BSA, Thermo-Fisher #11020-021) and a standard concentrations of antibiotics (penicillin/streptomycin and fungizone). The pre-mix can be stored in the refrigerator for up to two weeks.

At the time of culturing the following freshly thawed components were added to pre-mix. N2 Supplement (Thermo-Fisher A13707-01) 1:250, EGF (20 ng/ml; Peprotech), hFGF (5 ng/ml; Bechman), and IGF-1 (10-20 ng/ml; Peprotech).

Harvesting and Culturing Primary Tumor Cells

Cells were harvested using standard protocols involving collagenase/ialuronidase/dispase digestion, washing in media and filtration through 100 μm cell strainers. Once the specimens were received, they are minced and cut into small pieces. These fragments were then resuspended in 5 ml of media in the presence of collagenase/dispase and ialuronidase (concentration depending upon manufacturer specification). The samples were placed in a 37° C. incubator and gently swirled manually every 15 minutes. Total incubation time was about 1-1.5 hours or less.

The cells from the incubation step were then centrifuged, pelleted and re-suspended in the complete media described above and placed on the plates prepared as described above. The amount of media with which to re-suspend the cells is dependent upon the source of samples and the thickness of the pellet. For example, from a large-sized surgical sample, or a 300-400 ml pleural effusion, several cultures (six 6-well plates and one 10 cm plate) can be started. The day after the culture was initiated, the supernatant and all unattached cells were gently removed from the initial plates and transferred to fresh plates. This transfer procedure can generate additional attached cells, which were eventually pooled together with the first set of attached cells.

In another experiment, surgical samples of primary lung cancers from patients were minced and digested with dispase (50 u/ML) in DMEM media containing 5% serum for 1 hour. Pleural effusions were centrifuged, washed once in PBS and then plated. All primary cultures were initially grown on 6-well plates pre-coated with 2% GELTREX™ (Life technologies) and 1% MAXGEL™ (Sigma Aldrich). Unlike GELTREX™, MAXGEL™ is a humanized matrix, native and non-denatured.

The inclusion of MAXGEL™ in the system may or may not enhance the initial attachment of primary tumor cells. The day after plating, the media containing unattached cells, was transferred to a fresh plate, in most of cases generating new attached cultures. The media (Stem Cell media) is the DMEM/F12 (Thermo-fisher #11320-033 basic media) supplemented with the following components: Insulin-Transferrin-Sodium selenite (ITS; 1:1000; Sigma #11884); Glutamine (Thermo-fisher) to 4 mM final concentration; Sodium Pyruvate (Thermo-Fisher) to 2 mM final concentration; Rock Inhibitor (Y-27632) 10 micrograms/ml; 2-5% Knock Out Serum Replacement (KNOSR, Thermo-fisher #10828-028); 0.5% ALBUMAX™ (Lipid enriched BSA; Thermo-fisher #11020-021); N2 Supplement (Thermo-fisher) 1:250; EGF (5 ng/ml; Peprotech); hFGF (20 ng/ml; Bechman); IGF-1 (Peprotech; 10-20 ng/ml). The matrix was replaced every two days. At each passage duplicate cultures were generated by plating an aliquot of the GMSC-grown cells in regular attachment plates grown in DMEM and serum. This switch could often be achieved at around passage 7-10. The stromal component (mostly fibroblasts) was eliminated in early cultures with differential trypsinization (fibroblasts are more resistant to trypsin) and by growing the cells in low attachment plates and in Stem Cell Media for two or three passages. Fibroblasts are unable to form spheres while epithelial cells are enriched in these conditions. Cells can be frozen in stem cell media (or in DMEM media) and are re-started on matrix-coated plates.

Cells were passaged with Accutase (Life technologies/Thermofisher). Once growth is established in these conditions by at least two passages, the cells can be cultured in GELTREX™ alone (1.5-to-2.5% concentration), and can also be grown in DMEM media with standard cell culture plates.

### Example 2

### Cell Markers of Conditionally Immortalized Cancer Cells

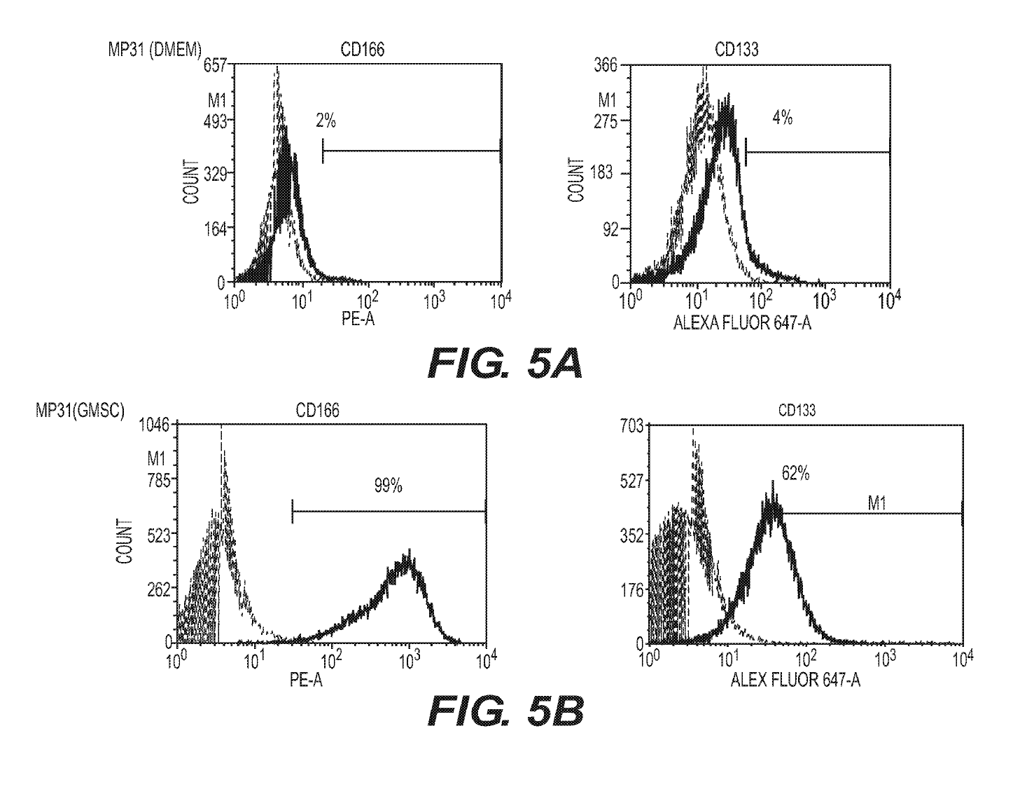

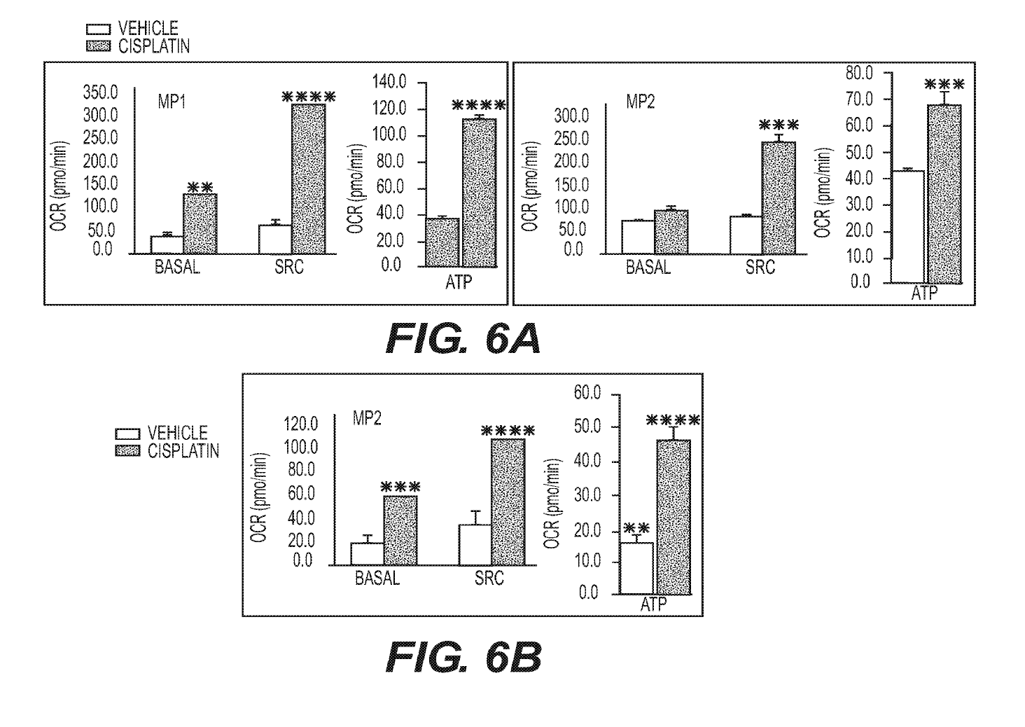

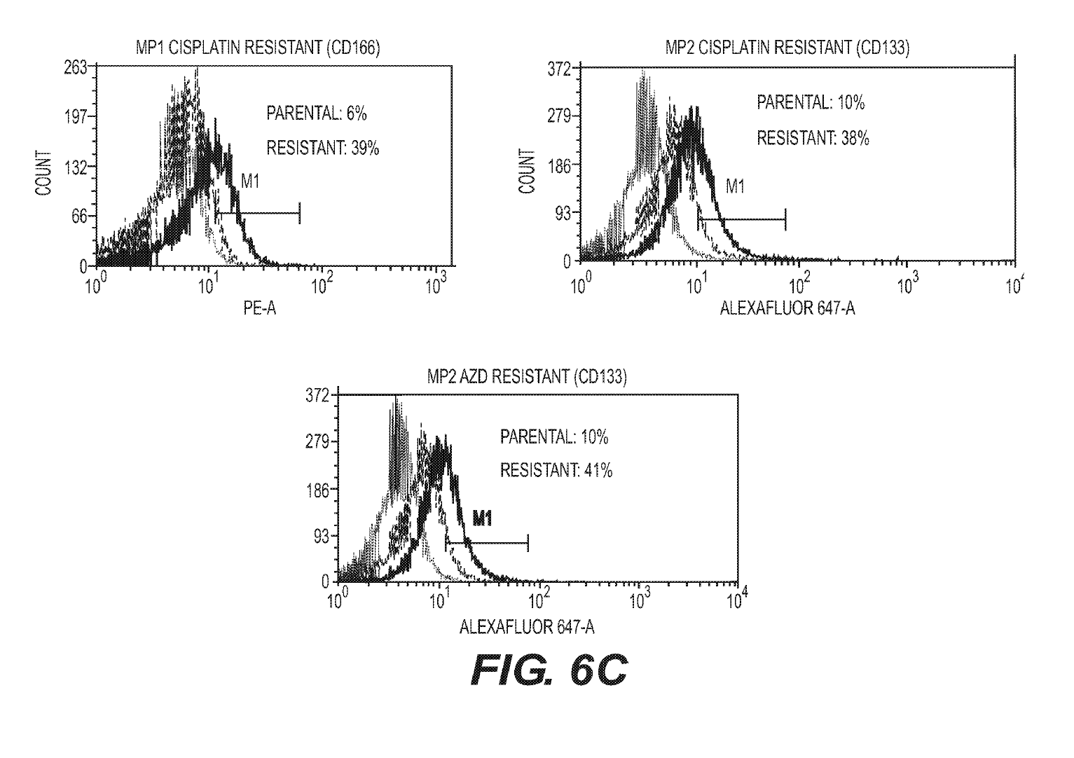

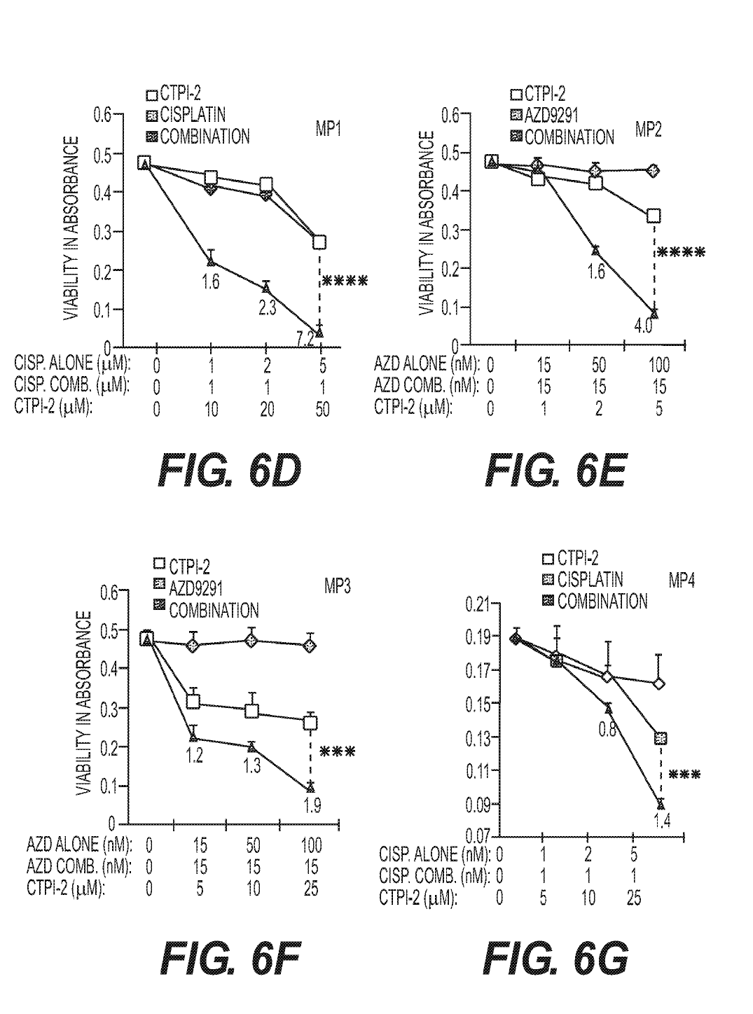

Cells were detached with Accutase, incubated with the specific antibodies for CD166 and CD133 in PBS containing 5% BSA for 30 minutes at room temperature, and analyzed by Flow Cytometry (FACS). Results are shown in FIG. 5 for FACS analysis of two stem cell markers, CD166 and CD133, in MP31 cells grown in canonical conditions, specifically in the presence of serum in DMEM media (A) or in GMSC (B). FIG. 5 shows significant enrichment of CSCs in GMSC conditions, but not in DMEM.

1-26. (canceled)

27. A composition comprising fibroblast growth factor (FGF), epithelial growth factor (EGF), insulin growth factor-1 (IGF-1), insulin, progesterone, transferrin, putrescine, pyruvate, albumin, selenite, thiamine, glutathione, ascorbic acid, and at least one Rho kinase (ROCK) inhibitor.

28. The composition of claim 27, wherein the composition further comprises glucose.

29. The composition of claim 28, wherein the composition further comprises at least one amino acid.

30. The composition of claim 29, wherein the composition comprises at least one amino acid selected from the group consisting of glycine, histidine, isoleucine, methionine, phenylalanine, praline, hydroxyproline, serine, threonine, tryptophan, tyrosine and valine.

31. The composition of claim 27, wherein the composition does not comprise animal serum.

32. The composition of claim 27, wherein the composition further comprises a base cell culture medium.

33. The composition of claim 27, wherein the ROCK inhibitor is an inhibitor of Rho kinase inhibitor 1 (ROCK 1), Rho kinase inhibitor 2 (ROCK 2) or both.

34. The composition of claim 33, wherein the ROCK inhibitor is selected from the group consisting of Y-27632, HA1100, HA1077, Thiazovivin, and GSK429286.

35. The composition of claim 33, wherein the ROCK inhibitor is an RNA interference (RNAi) molecule specific for ROCK 1, ROCK 2 or both.

36. A cell culture system comprising a composition according to claim 27 and a culture vessel.

37. The cell culture system of claim 36, wherein the culture vessel comprises extracellular matrix (ECM) components.

38. The cell culture system of claim 37, wherein at least a portion of the ECM components are human-derived components.

39. The cell culture system of claim 38, wherein the human-derived ECM components are selected from the group consisting of collagens, laminin, fibronectin, tenascin, and elastin.

40. The cell culture system of claim 39, wherein at least a portion of the ECM components are not human-derived components.

41. The cell culture system of claim 40, wherein the non-human-derived ECM components are entactin, heparan sulfate proteoglycan, or a combination thereof

42. A method of culturing cells isolated from subject biopsies, the method comprising placing the isolated cells in the cell culture system according to claim 36.

43. A population of conditionally immortalized cancer stem cells (CSCs).

44. A method of stimulating the growth of cancer stem cells (CSCs), the method comprising placing the cells in the cell culture system according to claim 36, whereby culturing the CSCs in the cell culture system will stimulate the growth of the CSCs.

45. A method of identifying a candidate cancer treatment for a subject in need of a treatment thereof, the method comprising
a) obtaining cells isolated from a biopsy from the subject,
b) culturing the isolated cells in the cell culture system according to claim 36 to produce a population of cancer cells in vitro,
c) determining a response profile of at least a portion of the cancer cells in vitro, and
d) identifying a candidate treatment for the subject based on the determined response profile.

46. The method of claim 45, wherein the response profile is at least partially determined by one or more of the following:
(i) identifying the sequence of at least one portion of DNA extracted from the cancer cells in vitro;
(ii) identifying at least one mRNA that is produced in the cancer cells in vitro;
(iii) identifying at least one mRNA that is not produced in the cancer in vitro;
(iv) identifying one or more proteins that the cancer cells in vitro express;
(v) identifying one or more proteins that the cancer cells in vitro do not express; and
(vi) subjecting the cancer cells in vitro to a therapeutic agent.
